(19) United States
(12) Patent Application Publication
(10) Pub. No.: US 2025/0325978 A1
(43) Pub. Date: Oct. 23, 2025

(54) PHOTOPATTERNED HYDROGELS

(71) Applicant: Georgia Tech Research Corporation, Atlanta, GA (US)

(72) Inventors: Ankur SINGH, Atlanta, GA (US); Manuel Quinones PEREZ, Atlanta, GA (US); Christopher R. CARLSON, Atlanta, GA (US); Andres J. GARCIA, Atlanta, GA (US); Zhe ZHONG, Atlanta, GA (US); Ana MORA BOZA, Atlanta, GA (US)

(21) Appl. No.: 18/870,904

(22) PCT Filed: Jun. 2, 2023
(86) PCT No.: PCT/US2023/067847

§ 371 Date: Dec. 2, 2024

Related U.S. Application Data
(60) Provisional application No. 63/348,191, filed on Jun. 2, 2022.

(51) Int. Cl. B01L 3/00; C07K 1/107; C12M 1/00; C12M 3/06

(52) U.S. Cl. CPC B01L 3/502707; B01L 3/502715; C07K 1/1077; C12M 23/16; C12M 29/10; B01L 2200/027; B01L 2200/12; B01L 2300/069

(57) ABSTRACT

Disclosed herein photopatterned hydrogels, hydrogel-based microfluidic devices, methods of making hydrogel-based microfluidic devices, and methods of using hydrogel-based microfluidic devices.

### CROSS-REFERENCE TO RELATED APPLICATION

This application claims the benefit of U.S. Provisional Application 63/348,191, filed on Jun. 2, 2022, the contents of which are hereby incorporated in their entirety.

### ACKNOWLEDGEMENT OF GOVERNMENT SUPPORT

This invention was made with government support under Grant No. DMR1847488 awarded by the National Science Foundation. The government has certain rights in the invention.

### REFERENCE TO SEQUENCE LISTING

The Sequence Listing submitted Jun. 2, 2023, as a text filed named “10034-178WO1_ST26.xml” created Jun. 1, 2023, and having a file size of 56,962 bytes is hereby incorporated by reference pursuant to 37 C.F.R. § 1.52(e)(5).

### FIELD OF THE INVENTION

The disclosure relates to photopatterned hydrogels, hydrogel-based microfluidic devices, methods of making hydrogel-based microfluidic devices, and methods of using hydrogel-based microfluidic devices.

### BACKGROUND

Engineering functional microenvironments together for three-dimensional (3D) tissue models is necessary to recapitulate key features of native tissues and organs. In native microenvironments, cells show precise spatial 3D arrangements, ensuring healthy functionalities of the tissues (i.e. cell-cell signaling, cell-matrix signaling and remodeling, transport of nutrients and oxygen, substance exchange, etc). Hydrogels are 3D polymeric network structures that can retain large amounts of water, providing unique environments for mimicking native tissues. Hydrogels show excellent cytocompatibility and permeability due to their hydrophilic and porous network, which can facilitate the transport of nutrients and biomolecules, being widely utilized for tissue manufacturing applications.

Recent advances in biomaterials and tissue engineering have focused on the development of new biofabrication techniques that can provide complex geometries to recreate functional microenvironments. A growing awareness of the limitations of traditional 2D cell culture systems gave rise to an expanding interest in 3D culture systems with perfusable microchannels that can facilitate cell survival and enhance tissue functionalities, moving towards more physiologically relevant in vitro tissue models. As such, perfusable microchannels in hydrogels are attractive platforms to recreate functional and intricate native networks (e.g. cardiovascular, lymphatic), enhance the transport of nutrients, oxygen and waste more efficiently, and support diverse multicell activities. These features are essential for advances in the organ-on-a-chip field and tissue modeling applications. While progress has been made regarding biofabrication techniques to develop intricate channels and geometries in hydrogels, existing methodologies are usually very time consuming, and they require from a specific training and expensive/sophisticated equipment.

There remains a need for improved systems and methods for preparing microfluidic devices. There remains a need for improved systems and methods for preparing microfluidic devices with highly intricate perfusable channels. The remains a need for improved systems and methods for rapidly preparing microfluidic devices using techniques that do not require specialized training and/or equipment.

### BRIEF DESCRIPTION OF THE FIGURES

FIG. 1A depicts a photopatterning approach to fabricate in situ perfusable microchannels in PEG-4aNB hydrogels and hydrogel characterization; hydrogel components comprising 4-arm branched amide norbornene PEG (PEG-4aNB) macromer, RGD as adhesive ligand, DTT or PEG-DT as crosslinking agent, and LAP as photoinitiator. Photopatterning strategy used for the fabrication of perfusable microchannels in hydrogels by injecting PEG-4aNB solution in a microfluidic device and applying UV light for in situ photopolymerization through a photomask.

FIG. 1B depicts swelling percentages for PEG-4aNB hydrogels fabricated with 5 and 20 kDa macromer sizes at 10 and 12 wt %, using DTT or PEG-DT as crosslinking agents.

FIG. 1C depicts storage and loss modulus of PEG-4aNB hydrogels fabricated with 5 and 20 kDa macromer sizes at 10 and 12 wt %, using DTT or PEG-DT as crosslinking agents after swelling. N=5, *p<0.05, **p<0.005, ***p=0.0002, ****p<0.0001. Two-way ANOVA analysis was used to detect significant differences among groups.

FIG. 2A depicts examples of photopatterned features in PEG-4aNB hydrogels and resolution characterization with bright field images of photomask transparencies used to generate photopatterns, and the resultant photopatterned PEG-4aNB hydrogels. Scale bar: 1 mm when not indicated.

FIG. 2B depicts shape fidelity of the photopatterned hydrogel relative to the corresponding photomask, in function of the photopatterned channel diameter; mean±SD, n=10 measurements per design.

FIG. 2C depicts fluorescence images of different photopatterned microchannels generated in PEG-4aNB hydrogels perfused with 70 kDa dextran molecule.

FIG. 3A depicts bright field images of photopatterning of 3 independent microchannels in PEG-4aNB hydrogels. Scale bar: 500 μm.

FIG. 3B depicts fluorescence intensity quantification of 70 and 3 kDa dextran molecules perfused over time (mean±SD, n=5).

FIG. 3C depicts fluorescence pictures of microchannels perfused with 70 and 3 kDa dextran molecules over time.

FIG. 3D depicts the photomask design used to create the photopatterned hydrogel. The square indicates the area where the fluorescent pictures in FIG. 3C where taken over time.

### DETAILED DESCRIPTION

Before the present methods and systems are disclosed and described, it is to be understood that the methods and systems are not limited to specific synthetic methods, specific components, or to particular compositions. It is also to be understood that the terminology used herein is for the purpose of describing particular embodiments only and is not intended to be limiting.

As used in the specification and the appended claims, the singular forms “a,” “an” and “the” include plural referents unless the context clearly dictates otherwise. Ranges may be expressed herein as from “about” one particular value, and/or to “about” another particular value. When such a range is expressed, another embodiment includes—from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint.

“Optional” or “optionally” means that the subsequently described event or circumstance may or may not occur, and that the description includes instances where said event or circumstance occurs and instances where it does not.

Throughout the description and claims of this specification, the word “comprise” and variations of the word, such as “comprising” and “comprises,” means “including but not limited to,” and is not intended to exclude, for example, other additives, components, integers or steps. “Exemplary” means “an example of” and is not intended to convey an indication of a preferred or ideal embodiment. “Such as” is not used in a restrictive sense, but for explanatory purposes.

Disclosed are components that can be used to perform the disclosed methods and systems. These and other components are disclosed herein, and it is understood that when combinations, subsets, interactions, groups, etc. of these components are disclosed that while specific reference of each various individual and collective combinations and permutation of these may not be explicitly disclosed, each is specifically contemplated and described herein, for all methods and systems. This applies to all aspects of this application including, but not limited to, steps in disclosed methods. Thus, if there are a variety of additional steps that can be performed it is understood that each of these additional steps can be performed with any specific embodiment or combination of embodiments of the disclosed methods.

Compounds disclosed herein may be provided in the form of physiologically acceptable salts. Examples of such salts are acid addition salts formed with inorganic acids, for example, hydrochloric, hydrobromic, sulfuric, phosphoric, and nitric acids and the like; salts formed with organic acids such as acetic, oxalic, tartaric, succinic, maleic, fumaric, gluconic, citric, malic, methanesulfonic, p-toluenesulfonic, napthalenesulfonic, and polygalacturonic acids, and the like; salts formed from elemental anions such as chloride, bromide, and iodide; salts formed from metal hydroxides, for example, sodium hydroxide, potassium hydroxide, calcium hydroxide, lithium hydroxide, and magnesium hydroxide; salts formed from metal carbonates, for example, sodium carbonate, potassium carbonate, calcium carbonate, and magnesium carbonate; salts formed from metal bicarbonates, for example, sodium bicarbonate and potassium bicarbonate; salts formed from metal sulfates, for example, sodium sulfate and potassium sulfate; and salts formed from metal nitrates, for example, sodium nitrate and potassium nitrate.

Disclosed herein are hydrogel-based microfluidic devices, methods of making hydrogel-based microfluidic devices, and methods of using hydrogel-based microfluidic devices in cell culture, drug discovery, and regenerative medicine.

In one aspect, the disclosure relates to methods of making microfluidic devices by selectively irradiating a hydrogel-precursor aqueous composition to selectively crosslink desired portions of the hydrogel-precursor. The uncrosslinked hydrogel-precursor composition may then be removed to provide a microfluidic device with well-defined inlets, channels, reservoirs, outlets, and the like.

In certain implementations, the hydrogel-precursor composition includes a multi-armed norbornenyl compound having the formula:

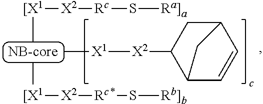

wherein NB-core represents the core of the multi-armed norbornenyl compound;
X¹ is in each case independently selected from null or a hydrophilic polymer;
X² is in each case independently selected from null or a linker;
R^{c} is in each case independently a divalent carbocyclic group;
R^{c*} is in each case independently a divalent carbocyclic group;
S—R^{a} is in each case independently an adhesion peptidyl group, the adhesion peptidyl group comprising a cysteine residue conjugated to R^{c};
R^{b} is in each case independently a biomolecule;
a is 0-4, 1-2, 1-3, 1-4, 2-3, 2-4, 3-4, 0, 1, 2, 3, or 4;
b is 0-4, 1-2, 1-3, 1-4, 2-3, 2-4, 3-4, 0, 1, 2, 3, or 4;
c is 2-8, 2-6, 2-4, 3-6, 5-8, 6-8, 2, 3, 4, 5, 6, 7, or 8; and
a multi-armed thiol compound having the formula:

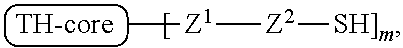

wherein TH-core represents the core of the multi-armed thiol compound;
Z¹ is null or a hydrophilic polymer;
Z² is null or a linker; and
m is 2-8, 2-6, 2-4, 3-6, 5-8, 6-8, 2, 3, 4, 5, 6, 7, or 8.

The hydrogel-precursor composition may further include an adhesion peptide as a separate compound. When a is 0, the hydrogel precursor includes an adhesion peptide as a separate compound. In some implementations the adhesion peptide includes at least one cysteine residue for conjugating to the norbonenyl group.

Whether present as the R^{a} group or a separate compound, the adhesion peptide can include at least one RGD sequence. In some implementations, the adhesion peptide (whether as R^{a} or a separate compound) includes: GRGDSPC (SEQ. ID 1), CRGDS(SEQ. ID 2), CRGDSP (SEQ. ID 3), CPHSRN (SEQ. ID 4), CGWGGRGDSP (SEQ. ID 5), CGGSIDQVEPYSSTAQ (SEQ. ID 6), CGGRNIAEIIKDI (SEQ. ID 7), CGGDITYVRLKF (SEQ. ID 8), CGGDITVTLNRL (SEQ. ID 9), CGGRYVVLPR (SEQ. ID 10), CGGKAFDITYVRLKF (SEQ. ID 11), CGGEGYGEGYIGSR (SEQ. ID 12), CGGATLQLQEGRLHFXFDLGKGR, wherein X=NIe (SEQ. ID 13), CGGSYWYRIEASRTG (SEQ. ID 14), CGGGEFYFDLRLKGDKY (SEQ. ID 15), CKGGNGEPRGDTYRAY (SEQ. ID 16), CKGGPQVTRGDVFTMP (SEQ. ID 17), CGGNRWHSIYITRFG (SEQ. ID 18), CGGASIKVAVSADR (SEQ. ID 19), CGGTTVKYIFR (SEQ. ID 20), CGGSIKIRGTYS (SEQ. ID 21), CGGSINNNR (SEQ. ID 22), CGGSDPGYIGSR (SEQ. ID 23), CYIGSR (SEQ. ID 24), CGGTPGPQGIAGQGVV (SEQ. ID 25), CGGTPGPQGIAGQRVV (SEQ. ID 26), CGGMNYYSNS (SEQ. ID 27), CGGKKQRFRHRNRKG (SEQ. ID 28), CRGDGGGGGGGGGGGGGPHSRN (SEQ. ID 29), CPHSRNSGSGSGSGSGRGD (SEQ. ID 30), acetylated-GCYGRGDSPG (SEQ. ID 31), ((GPP)5GPC) (SEQ. ID 32), CRDGS (SEQ. ID 33), cyclic RGD{Fd}C (SEQ. ID 34), CGGRKRLQVQLSIRT (SEQ. ID 35), CIKVAV (SEQ. ID 36), CGGAASIKVAVSADR (SEQ. ID 37), CGGKRTGQYKL (SEQ. ID 38), CGGTYRSRKY (SEQ. ID 39), CGGYGGGP(GPP)5GFOGERPP(GPP)4GPC (SEQ. ID 40), CGGKRTGQYKLGSKTGPGQK (SEQ. ID 41), QAKHKQRKRLKSSC (SEQ. ID 42), SPKHHSQRARKKKNKNC (SEQ. ID 43), CGGXBBXBX, wherein B=basic residue and X=hydropathic residue (SEQ. ID 44), and CGGXBBBXXBX, wherein B=basic residue and X=hydropathic residue (SEQ. ID 45), or a combination thereof.

In certain implementations, a is not 0, e.g., a is 1 or 2, and R^{c} has the formula:

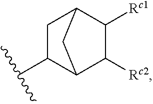

wherein one of R^{c1} and R^{c2} is S—R^{a}, the other of R^{c1} and R^{c2} is H, and the squiggly line represents the point of attachment to X².

In some implementations, b is 0. In other implementations, b is not 0, e.g., b is 1 or 2. When b is not 0, R^{c*} has the formula:

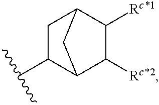

wherein one of R^{c*1} and R^{c*2} is S—R^{b}, the of R^{c*1} and R^{c*2} other is H, and the squiggle line represents the point of attachment to X².

When present, R^{b} can include various biopolymers and biomolecules. In certain implementations, R^{b} is a nucleic acid, polysaccharide, protein, lipid, tracer compound, aptamer, steroid, signaling molecule, or a combination thereof.

In some implementations, X¹ is (CH₂CH₂O)ₓ, wherein x is in each case independently selected from 1-500, 5-50, 5-25, 10-25, 10-30, 25-50, 25-75, 50-100, 75-150, 100-250, or 250-500. In other implementations, X¹ is null.

In certain implementations, X² is in each case independently selected from null, C(═O), CH₂CH₂, CH₂CH₂NH, or CH₂CH₂NHC(═O), preferably X² is in each case C(═O). The skilled person will recognize that compounds in which X² is in each case C(═O) can be derived from 5-norbornene-2-carboxylic acid.

The multi-armed norbornenyl compound may be derived from various polyalcohols, for instance pentaerythritol, dipentaerythritol, tripentaerythritol, glycerol, triglycerol, and the like. In certain implementations, the NB-core has the formula:

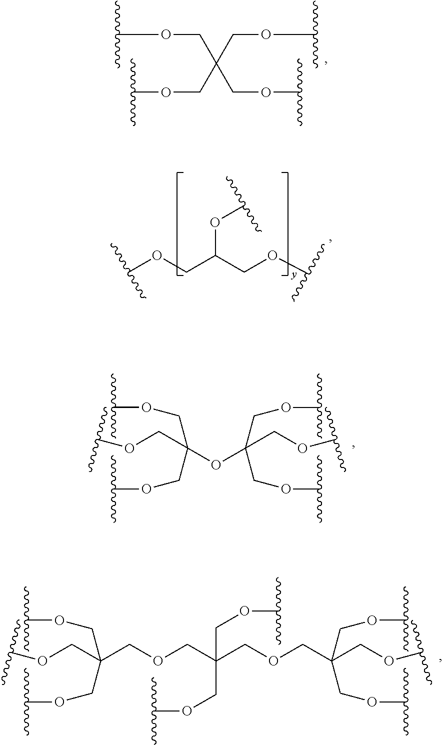

wherein y is 1-6, and each squiggly line represents the point of attachment to X¹.

In some implementations, Z¹ is in each case independently selected from null or (CH₂CH₂O)_{z}, wherein z is in each case independently selected from 1-500, 5-50, 5-25, 10-25, 10-30, 25-50, 25-75, 50-100, 75-150, 100-250, or 250-500. In certain implementations, Z¹ is in each case null.

In some implementations, Z² is null, C(═O), CH₂CH₂, CH₂CH₂NH, or CH₂CH₂NHC(═O), preferably null or CH₂CH₂.

In some implementations, TH-core can have the formula:

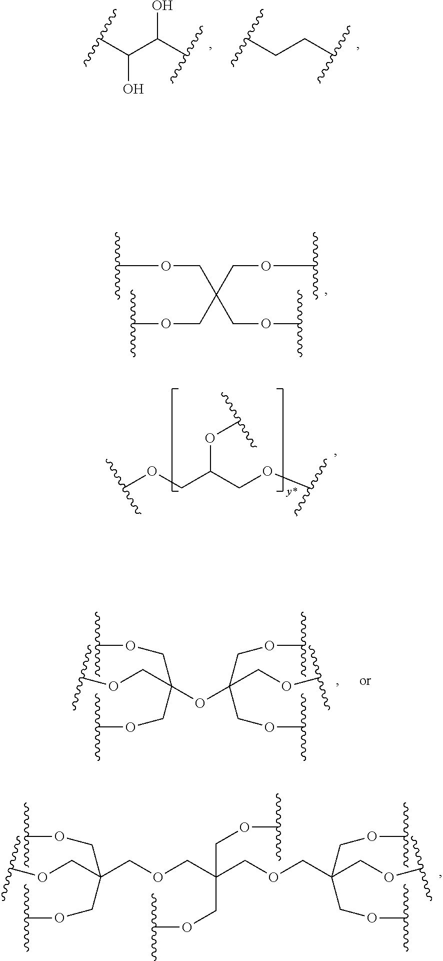

wherein y* is 1-6, and each squiggly line represents the point of attachment to Z¹.

The multi-armed norbornenyl compound may be present in the aqueous hydrogel precursor composition in an amount from 1-25 wt. %, from 2-20 wt. %, from 5-15 wt. %, from 5-10 wt. %, from 5-7.5 wt. %, from 7.5-12.5 wt. %, from 7.5-10 wt. %, from 10-12.5 wt. %, from 10-15 wt. %, from 12.5-15 wt. %, from 15-17.5 wt. %, from 15-20 wt. %, or from 17.5-20 wt. %.

The multi-armed thiol compound may be present in the aqueous hydrogel precursor composition in an amount sufficient to crosslink the multi-armed norbornenyl compound. In certain implementation the molar ratio of [SH groups] in the multi-armed thiol compound to the [norbornenyl groups] in the multi-armed norbornenyl compound can be from 10:1 to 1:10, from 5:1 to 1:5, from 10:1 to 1:1, from 10:1 to 5:1, from 5:1 to 2.5:1, from 5:1 to 1:1 from 2.5:1 to 1:1, from 2.5:1 to 1:2.5, from 1:1 to 1:2.5, from 1:1 to 1:5, from 1:1 to 1:10, 1:2.5 to 1:5, or from 1:5 to 1:10.

In some implementations, the multi-arm thiol compound may be present in the aqueous hydrogel precursor composition at a concentration from 1-100 mM, from 1-50 mM, from 1-25 mM, from 1-10 mM, from 5-10 mM, from 5-15 mM, from 5-25 mM, from 10-25 mM, from 25-50 mM, from 25-75 mM, from 50-75 mM, from 50-100 mM, or from 75-100 mM.

When provided as a separate compound, the adhesion peptide may be present in the aqueous hydrogel precursor composition at a concentration from 1-50 mM, from 1-25 mM, from 1-10 mM, from 1-5 mM, from 2.5-5 mM, from 2.5-10 mM, from 5-10 mM, from 5-15 mM, from 10-25 mM, from 10-50 mM, or from 25-50 mM. In some implementation the molar ratio of the adhesion peptide and multi-arm thiol compound is from 1:1 to 1:20, from 1:1 to 1:10, from 1:1 to 1:5, from 1:5 to 1:10, from 1:5 to 1:15, from 1:10 to 1:15, from 1:10 to 1:20, or from 1:15 to 1:20.

In certain implementations, the hydrogel precursor composition will include one or more photoinitiators. In certain implementations, the photoinitiator can be a salt or ester of phenyl-2,4,6-trimethylbenzoylphosphinate, e.g., lithium phenyl-2,4,6-trimethylbenzoylphosphinate or ethyl phenyl-2,4,6-trimethylbenzoylphosphinate, a benzoyl formate like methyl benzoyl formate, 2,2′-azobis[2-methyl-N-(2-hydroxyethyl) promionamide. The photoinitiator can be provided in the aqueous hydrogel precursor composition at a concentration from 0.1-10 mM, from 0.1-5 mM, from 0.1-2.5 mM, from 0.5-1.5 mM, from 1-3 mM, or from 2-5 mM.

In certain implementations, the selective irradiation may be performed by providing a mask between the hydrogel-precursor composition and light source, wherein the mask has transparent and opaque portions, the opaque portions defining the portions of the hydrogel-precursor composition that are not crosslinked. In other implementations, the selective crosslinking may be performed using photolithographic processes wherein a controllable laser is used to selectively irradiate portions of the hydrogel-precursor composition without using a mask.

In certain implementations, the selective irradiation may be performed using a UW lamp applied at an intensity from 1-250 mW/cm², from 50-250 mW/cm², from 50-200 mW/cm², from 50-150 mW/cm², from 50-100 mW/cm², from 100-150 mW/cm², from 75-125 mW/cm², from 1-50 mW/cm², or from 25-75 mW/cm². The composition can be irradiated for a period from 0.1-3 seconds, from 0.5-3 seconds, from 0.5-2 second, from 0.5-1.5 seconds, from 0.5-1 seconds, or from 1-1.5 seconds.

Also disclosed herein are microfluidic devices. In certain implementations, the device is a multilayer device including at least two substrate layers and a microfluidic layer. The microfluidic layer includes the crosslinked hydrogel compositions disclosed herein and defines at least one channel configured to receive a liquid. The substrate layers may be any transparent material through which the crosslinking radiation may be passed. Exemplary transparent materials include polydimethylsiloxanes (PDMS). In certain implementations the device includes a bottom substrate layer, a top substrate layer, and a microfluidic layer disposed there between. In some implementations, the device includes a base and a cover. The base can include only the bottom substrate layer, or it may include the bottom substrate layer and further layers of more durable material. Similarly, the cover can include only the top substrate layer, or it may include the top substrate layer and further layers of more durable material.

The terms “bottom,” “base,” “top,” and “cover” relate to the bias of gravity acting upon a liquid in the channel. The bottom substrate layer contacts one of the surfaces of the microfluidic layer and may be designated the bottom surface. The top substrate layer contacts the other of the surfaces of the microfluidic layer and may be designated the top surface. The channel extends from the bottom surface of the microfluidic layer to the top surface of the microfluidic layer. A length of the channel is measured along a longitudinal axis of the channel. A width of the channel is measured perpendicular to the longitudinal axis. A height of the channel is measured as the distance between the top surface and the bottom surface of the microfluidic layer and is perpendicular to both the length and the width of the channel.

In certain implementations, the channel is in fluid communication with at least two ports, such that liquid may be introduced into the channel through one port (an inlet port) and exit at another port (an outlet port) after being pumped through the channel. In certain implementations, the inlet port is defined by an aperture defined in the top substrate layer (or defined in the cover), which is configured to be placed in fluid communication with a pump to drive perfusion of liquid.

In certain implementations, the microfluidic layer includes a plurality of channels. Each of the channels may be in fluid communication with the same two ports, or each channel may be in fluid communication with a separate pair of ports. For example, in some implementations, a first channel or a first set of the channels is in fluid communication with a first pair of ports, and a second channel or a second set of the channels is in fluid communication with a second pair of ports. As another example, in some implementations, the device may include multiple channels, wherein each channel is in fluid communication with a separate inlet port, and each channel is in fluid communication with the same outlet port.

In certain implementations, the channel has a width of from 10-1,000 μm, from 10-500 μm, from 10-250 μm, from 10-100 μm, from 10-50 μm, from 50-100 μm, from 50-150 μm, from 100-250 μm, from 250-500 μm, or from 500-1,000 μm. In certain implementations, the microfluidic device includes a plurality of channels, each channel having a width independent of the other channels. Each of the channels may have a uniform width along its length, such that the width is always within 10%, 5%, or 2.5% of the specified distance. By controlling the width of each individual channel, the device provides selective transport of differently sized molecules across some, but not all channels. This is an important feature for organ-on-a-chip devices that mimic biological systems.

The microfluidic devices disclosed herein a characterized by excellent fidelity relative to the corresponding photomask used to prepare them. In certain implementations, the photopatterned hydrogel will have at least 75%, at least 80%, at least 85%, at least 90%, at least 95%, or at least 99% fidelity relative to the corresponding photomask. Fidelity can be assessed by calculating the deviation of the measurements for photopatterned features in comparison to the designs on the photomasks.

Also disclosed herein are methods of making cellular structures with the disclosed microfluidic devices. In certain implementations, the method includes the step of depositing a cell, cellular precursor, or combination thereof into a channel of the microfluidic device. The channel is then perfused with a nutrient mixture to produce the cellular structure. In some implementations the cell or cellular precursor is a tissue sample or stem cell. In the context of the disclosed method, a “cell” is not limited to a single cell and is inclusive of a plurality of cells. Exemplary stem cells include multipotent stem cells, pluripotent stem cells, totipotent stem cells, embryonic stem cells, extraembryonic fetal stem cells, amniotic stem cells.

In certain implementations the stem cell is an adult stem cell, for example hematopoietic stem cell, endothelial stem cell, intestinal stem cell, mammary stem cell, neural stem cell, or mesenchymal stem cell.

In certain implementations, the cellular structure is a vascular tissue, a germinal center, an organoid, a cell aggregate (either single cellular or multi-cellular), or a bacteria.

In some implementations the method can be used to produce a lung organoid, cerebral organoid, thyroid organoid, thymic organoid, testicular organoid, hepatic organoid, pancreatic organoid, gut organoid (i.e., intestinal organoid, gastric organoid, or lingual organoid), epithelial organoid, lung organoid, renal organoid, embryonic organoid, or cardiac organoid.

In certain implementations the channels of the microfluidic device are configured to function similarly or mimic the natural in vivo systems found in whole organs/living organisms. Because the channels of the microfluidic devices may be very precisely defined using the method disclosed herein, the device can recreate the intricate, functional networks found in natural tissues like cardiovascular tissue and lymphatic tissue. The multifluidic devices disclosed herein can be used to test the interaction of cellular structures with various endogenous and/or exogenous factors, for example growth factors, signaling molecules, proteins, drugs, toxins, antigens, and the like.

### Examples

The following examples are for the purpose of illustration of the invention only and are not intended to limit the scope of the present invention in any manner whatsoever.

The PDMS layers to fabricate the microfluidic devices were cast at a 10:1 mass ratio of elastomer:curing agent in plastic tissue culture dishes and cured at room temperature for 24 hours. The resulting PDMS layers were removed from the petri dishes and bonded to cover glass slides or PDMS using O₂ plasma. The master silicon wafer with the channel designs was prepared using SU8-2100 (Kayakuam) and soft lithography techniques in Marcus Inorganic Cleanroom of Georgia Tech. PDMS was cast to the master silicon wafer and heated to 70° C. for 4 hours. The resulting PDMS was bonded to the rest of PDMS/cover glass layers using O₂ plasma, and the final device was heated overnight at 70° C.

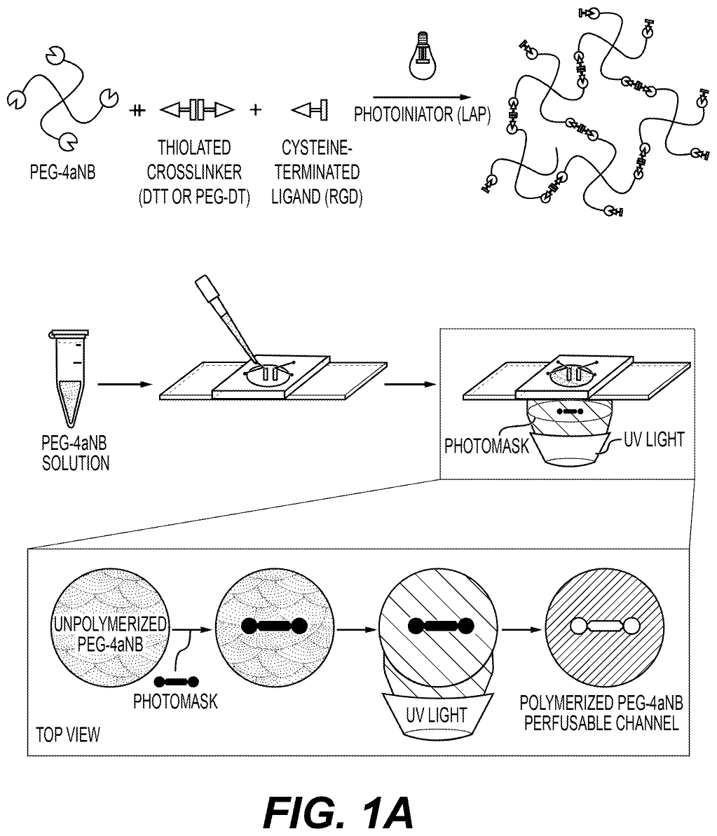

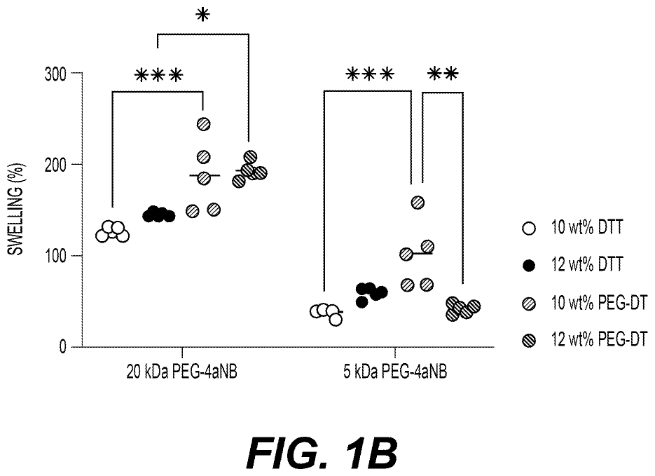

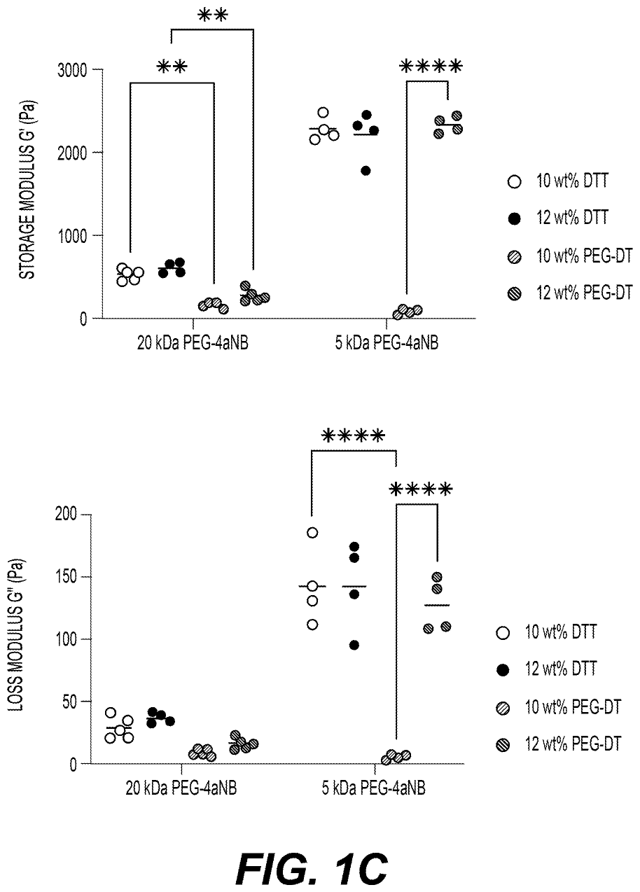

PEG-4aNB macromers of 5 kDa and 20 kDa molecular weight (Jenkem Technologies) were evaluated for hydrogel preparation at different concentrations using 1,4-dithiothreitol (DTT, Sigma) or hexa(ethylene glycol)-dithiol (PEG-DT, Sigma) as crosslinking agents. PEG-4aNB macromers at 10 or 12 wt. % were dissolved in 10 mM HEPES in PBS, containing 4.0 mM of RGD (GRGDSPC, Vivitide) as a cell adhesive ligand, 1.0 mM of lithium phenyl-2,4,6-trimethylbenzoylphosphinate (LAP, Tocris) as photoinitiator agent, and the corresponding concentration for the crosslinking agent: 36.20 mM or 7.55 mM for 5 kDa and 20 kDa macromer at 10 wt. %, respectively, and 43.84 mM or 9.46 mM 12 wt. % 5 kDa and 20 kDa at 12 wt. %, respectively. Thus, 120 μL of polymer solution was injected in the microfluidic device, consisting of a double sandwich elastomeric structure based on PDMS and coverslip substrate, previously bonded with O₂ plasma (FIG. 1A). After injection, the photomask with the designed features was placed on the bottom of the device (i.e. glass side) and UV light (OmniCure S2000 Spot UV Curing System, Excellitas Technologies) was applied at 100 mW/cm² for 0.8 seconds. The non-crosslinked polymer solution was removed by flushing PBS through the fabricated perfusable microchannels after photopolymerization. The photomask features were designed using AutoCAD® software (2023) and transparencies were prepared Mylar film by Arnet Pro.

To characterize the resolution of the photopattern technique and shape fidelity of the generated features, 120 μL polymer solution was photopolymerized in 35 mm glass bottom dishes with 10 mm micro-well (Cellvis) using the same approach described above. Crosslinked hydrogels were washed with 2 mL of PBS to remove the unpolymerized solution. Tile scan images were taken in EVOs (Ankur) and feature sizes were measured (n=8 per design) and compared to the photomask designs to determine the resolution and shape fidelity of the photopatterning technique. To analyze the perfusability and diffusion properties of the photopatterned microchannels, the hydrogel solution was injected and photopolymerized in the microfluidic device as previously explained, and a solution of Cy5.5-labeled dextran (25 μg/mL, 70 kDa, Nanocs) or Texas red-Dextran (25 μg/mL, 3 kDa, ThermoFisher) was flowed through the microchannels. Fluorescence images and tile scan images were taken 10× objective (CFI60 Plan Apochromat Lambda D, N.A. 0.45, W.D. 4.0 mm, F.O.V. 25 mm) in a Nikon Ti2-E motorized inverted microscope and AX-R Confocal System. Fluorescence quantification was performed with ImageJ.

Swelling and mechanical properties of hydrogels were examined for various weight percentages and crosslinking agents to assess for differences between the two macromer sizes. For swelling characterization, 20 μL of polymer solution was crosslinked, weighted after preparation (w₀), and incubated overnight in PBS at 37° C. (n=5 per condition). The following day, excess water was removed and the hydrogels were weighted again (w₁). The swelling percentage was calculated using the following equation: Swelling (%)=(w₁−wₒ)/wₒ*100.

The same crosslinked hydrogels were used for characterization of mechanical properties after overnight swelling in PBS. Rheological properties were measured using a cone and plate rheometer (2° cone with 10 mm diameter, MCR 302, Anton-Paar). Measurements were obtained by averaging the storage and loss modulus over an oscillatory frequency range of 1-10 Hz at 1.5% strain at 25° C. (linear viscoelastic range was determined using oscillatory strain amplitude sweep, n=5 per condition).

Human umbilical vein endothelial cells (HUVECs, Angioproteome) expressing green fluorescence protein (GFP+) at passage 3-5 were trypsinized for 5 min and resuspended to a final concentration of 4×10⁶ cell/mL in EGM™ endothelial cell growth medium (Lonza). The cell suspension was injected into the perfusable channels of the hydrogels and incubated for 2 hours to allow cell adhesion to channel surfaces. Microfluidic devices were then connected to a syringe pump (10-rack PHD ULTRA™ Syringe Pumps, Harvard Apparatus) at infusion mode at 1 μL/min. Syringes contained growth media with amphotericin B (2.50 μg/mL, Thermofisher), which was automatically infused through the microfluidic devices that were placed in an incubator at 37° C. and CO₂ (5%). Syringes with fresh media were replaced every 4 days.

Cell viability was analyzed after 2 and 7 days of culture under media perfusion. Cells were stained for 15 min with BioTracker 405 Blue Mitochondria dye at final concentration of 1 μM for live cells (Sigma) and TOTO™-3 Iodide dye at final concentration of 1 μM for dead cells (Thermofisher) to analyze cell viability (n=3). Fluorescence images and tile scan images were taken in Nikon Ti2-E motorized inverted microscope and AX-R Confocal System. Permeability barrier functionality was analyzed by perfusing Cy5.5-labeled dextran (25 μg/mL, 70 kDa, Nanocs) solution. Fluorescence pictures were taken 15 min after dextran perfusion.

Data was analyzed using GraphPad Prism 8 (Graph-Pad Software Inc., La Jolla, CA). Statistical tests are specified, and P values provided. For swelling and mechanical properties measurements, two-way ANOVA analysis was performed to study significant differences among macromer sizes, polymer concentrations and crosslinking agents. All data represented as means±standard deviations (SD).

Photopolymerizable PEG-4aNB was used to fabricate perfusable microchannels in hydrogels by applying UV light through a photomask with different microchannel designs. FIG. 1a describes the hydrogel components for the fabrication of perfusable channels using a custom-made microfluidic device. Our device consisted of a double sandwich elastomeric structure based on three different layers of PDMS and a coverslip glass. The first layer of PDMS had two independent sets of two channels that allowed the injection of polymer solution into a PDMS chamber. The second layer of PDMS consisted of two holes that were aligned to the ending of the channels of the first PDMS layer and was bonded to a third layer of PDMS containing a 10 mm diameter cavity, where the hydrogel solution was injected and photopolymerized. The three PDMS layers, together with the channels set, allowed the perfusion of the confined hydrogel when connected to an external pump system. After injection of the PEG-4aNB solution, a photomask transparency with the desired channel design was placed on the glass bottom side of the device and UV light was applied for 0.8 seconds through the photomask, allowing the photopolymerization of the polymer solution that was not shielded by the pattern design. The un-crosslinked polymer was then flushed by flowing PBS through the inlets/outlets. To determine the optimal hydrogel formulation for our photopatterning approach, we evaluated the swelling and mechanical properties of 8 different polymer formulations (FIG. 1B, 1C). We analyzed the swelling percentages of PEG-4aNB hydrogels fabricated with 5 kDa and 20 kDa macromers, observing that hydrogels fabricated with 5 kDa PEG-4aNB macromer showed a significantly reduced swelling in comparison to 20 kDa macromer at all polymer concentrations and for both DTT and PEG-DT crosslinkers (FIG. 1B). These swelling results showed excellent correlation with rheological measurements, obtaining significantly higher storage and loss modulus values for those hydrogel formulations that showed lower swelling percentages (FIG. 1C). Thus, 5 kDa PEG-4aNB at 10 wt. % crosslinked with DTT was selected as the optimal hydrogel formulation for our photopatterning approach, since low swelling degrees (38±5%) would lead to higher shape fidelity and resolution in the photopatterned features in the hydrogels.

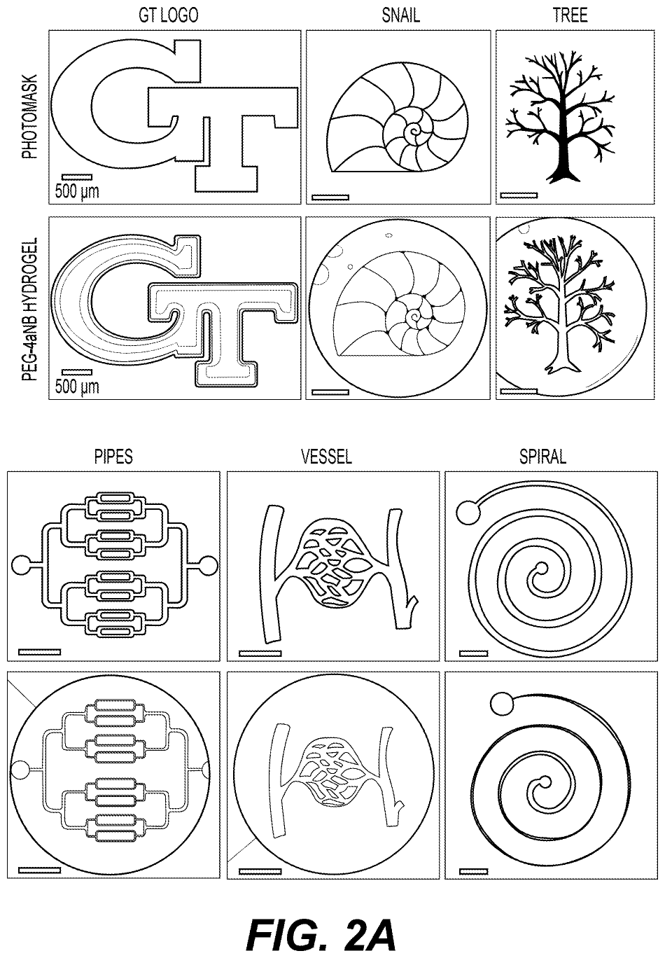

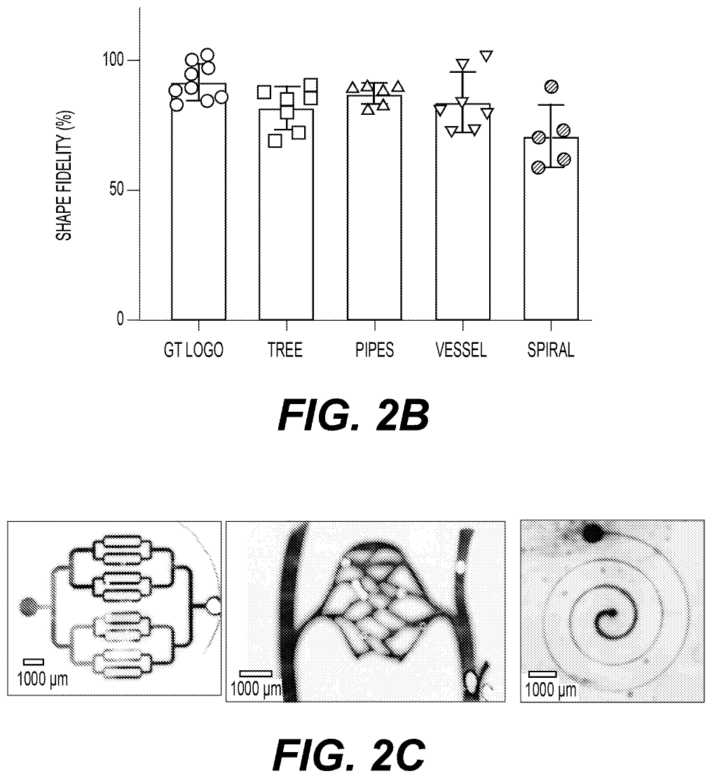

Different photomask transparencies with intricate and complex designs were tested to evaluate the versatility and resolution of the photopatterning approach in PEG-4aNB hydrogels (FIG. 2A). Excellent shape fidelity was observed for the all the photopatterned designs, obtaining values of 83±8% when compared to the features of the photomask (FIG. 2B). The resolution of the features was very high even for very complex designs with different angles, such as the snail or the tree design, being able to photopattern microchannels down to 25 μm. Moreover, the photopatterned channels demonstrated excellent perfusability against fluorescent dextran molecule (70 kDa), as it can be observed in FIG. 2C.

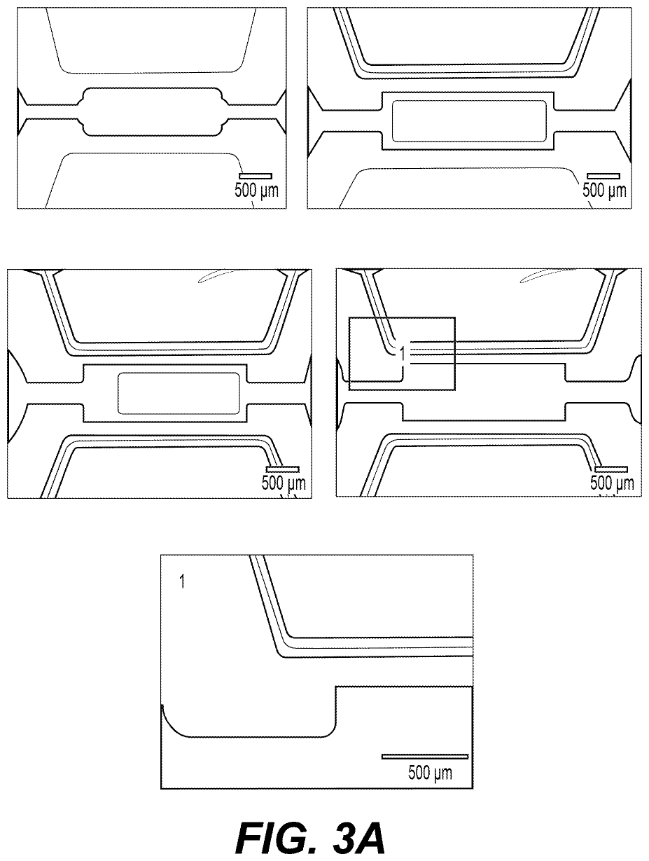

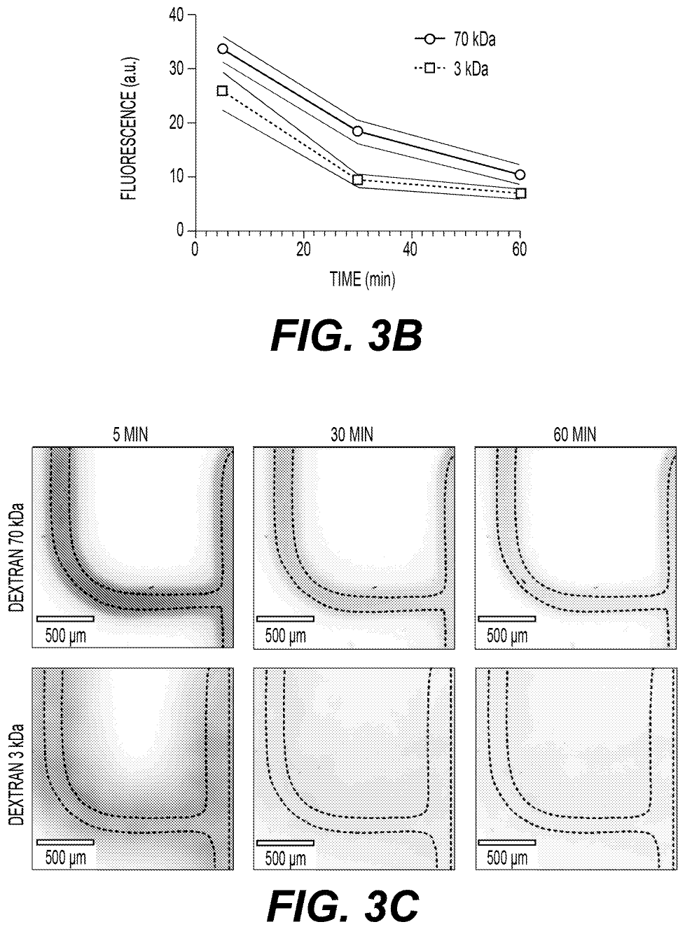

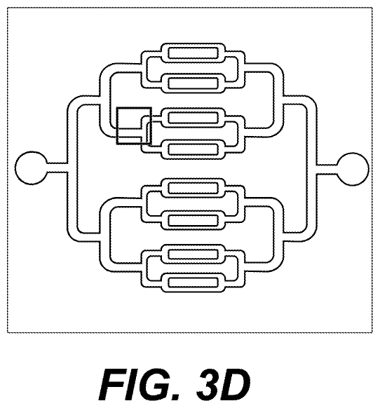

Our photopatterning technique allowed the fabrication of multiple and independent microchannels in PEG-4aNB hydrogels. FIG. 3A shows perfusion of a PEG-4aNB hydrogel photopatterned with three independent microchannels. Dyes of different colors were perfused through 3 independent channels without leaking between the consecutive channels. In addition, some dye diffusion into the hydrogel matrix could be observed after 5 min of injection, demonstrating the diffusion capacity of the hydrogels (FIG. 3A, magnification image 1). The diffusion properties of the photopatterned hydrogels were also assessed using dextran molecules with different molecular weights (i.e., 3 and 70 kDa). FIGS. 3B and 3C shows that fluorescence intensities inside the channel decreased over time for both dextran molecules as they diffused into the hydrogel matrix, demonstrating the diffusion capacity of the system. In addition, the diffusivity rate was dependent on the molecule size, observing a slower diffusion for 70 kDa dextran in comparison to 3 kDa (FIG. 3B, 3C). These results show the capacity of these systems to transport molecules of different sizes from the perfusable channels into the hydrogel matrix, and also between independent channels. This is an important property for organ-on-a-chip models that mimic biological tissues.

HUVECs were seeded in a single channel photopatterned PEG-4aNB hydrogel and connected to an external pump system to perfuse the device over time. Live/dead assay was performed after 2 and 7 days of culture. The microchannel surface exhibited near total coverage by HUVECs after 2 days of seeding and the cultured cells showed excellent viability with almost no dead cells. Healthy cell morphology and spreading were evident. We also evaluated the barrier functionality of the cultured HUVECs over time. In HUVEC-lined channels perfused with 70 kDa dextran molecule the tracer was maintained within the perfusable channel and did not diffuse into the hydrogel matrix.

After 7 days of culture under perfusion, HUVECs proliferated and colonized the complete surface of the microchannel, showing confluent endothelial monolayer with excellent viability and morphology. HUVEC-lined channels were perfused with 70 kDa dextran molecule. The fluorescent tracer was retained with the microchannel and confirmed barrier function for the cultured HUVECs.

HUVECs were also seeded and cultured under perfusion in a PEG-4aNB hydrogel photopatterned with a multichannel design. An increasing number of cells were observed after 7 days of perfusion, indicating the proliferation and colonization of the multichannel design by HUVECs.

The compositions and methods of the appended claims are not limited in scope by the specific compositions and methods described herein, which are intended as illustrations of a few aspects of the claims and any compositions and methods that are functionally equivalent are intended to fall within the scope of the claims. Various modifications of the compositions and methods in addition to those shown and described herein are intended to fall within the scope of the appended claims. Further, while only certain representative compositions and method steps disclosed herein are specifically described, other combinations of the compositions and method steps also are intended to fall within the scope of the appended claims, even if not specifically recited. Thus, a combination of steps, elements, components, or constituents may be explicitly mentioned herein or less, however, other combinations of steps, elements, components, and constituents are included, even though not explicitly stated. The term “comprising” and variations thereof as used herein is used synonymously with the term “including” and variations thereof and are open, non-limiting terms. Although the terms “comprising” and “including” have been used herein to describe various embodiments, the terms “consisting essentially of” and “consisting of” can be used in place of “comprising” and “including” to provide for more specific embodiments of the invention and are also disclosed. Other than in the examples, or where otherwise noted, all numbers expressing quantities of ingredients, reaction conditions, and so forth used in the specification and claims are to be understood at the very least, and not as an attempt to limit the application of the doctrine of equivalents to the scope of the claims, to be construed in light of the number of significant digits and ordinary rounding approaches

1-32. (canceled)

33. A method of making a crosslinked network, comprising:
1) selectively irradiating an aqueous composition comprising:
a) a multi-armed norbornenyl compound having the formula: 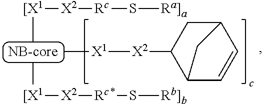
wherein NB-core represents the core of the multi-armed norbornenyl compound;
X¹ is in each case independently selected from null or a hydrophilic polymer;
X² is in each case independently selected from null or a linker;
R^{c} is in each case independently a divalent carbocyclic group;
R^{c+} is in each case independently a divalent carbocyclic group;
S—R^{a} is in each case independently an adhesion peptide, the adhesion peptide comprising a cysteine residue conjugated to R^{c};
R^{b} is in each case independently a biomolecule;
a is 0-4;
b is 0-4;
c is 2-8
b) a multi-armed thiol compound; having the formula: 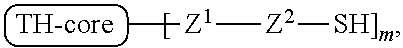
wherein TH-core represents the core of the multi-armed thiol compound;
Z¹ is null or a hydrophilic polymer;
Z² is null or a linker; and
m is 2-8;
c) optionally, an adhesion peptide having at least one cysteine residue;
wherein the selective irradiation crosslinks a portion of the composition to give a crosslinked network; and
2) removing the uncrosslinked composition from the crosslinked network.

34. The method of claim 33, wherein
R^{c} has the formula: 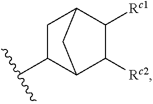
wherein one of R^{c1} and R^{c2} is S—R^{a}, and the other is H; and
R^{c*} has the formula: 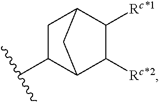
wherein one of R^{c*1} and R^{c*2} is S—R^{b}, and the other is H.

35. The method of claim 33, wherein the adhesion peptide/R^{a} comprises an RGD sequence.

36. The method of claim 33, wherein the adhesion peptide/R^{a} comprises GRGDSPC (SEQ. ID 1), CRGDS(SEQ. ID 2), CRGDSP (SEQ. ID 3), CPHSRN (SEQ. ID 4), CGWGGRGDSP (SEQ. ID 5), CGGSIDQVEPYSSTAQ (SEQ. ID 6), CGGRNIAEIIKDI (SEQ. ID 7), CGGDITYVRLKF (SEQ. ID 8), CGGDITVTLNRL (SEQ. ID 9), CGGRYVVLPR (SEQ. ID 10), CGGKAFDITYVRLKF (SEQ. ID 11), CGGEGYGEGYIGSR (SEQ. ID 12), CGGATLQLQEGRLHFXFDLGKGR, wherein X=Nle (SEQ. ID 13), CGGSYWYRIEASRTG (SEQ. ID 14), CGGGEFYFDLRLKGDKY (SEQ. ID 15), CKGGNGEPRGDTYRAY (SEQ. ID 16), CKGGPQVTRGDVFTMP (SEQ. ID 17), CGGNRWHSIYITRFG (SEQ. ID 18), CGGASIKVAVSADR (SEQ. ID 19), CGGTTVKYIFR (SEQ. ID 20), CGGSIKIRGTYS (SEQ. ID 21), CGGSINNNR (SEQ. ID 22), CGGSDPGYIGSR (SEQ. ID 23), CYIGSR (SEQ. ID 24), CGGTPGPQGIAGQGW (SEQ. ID 25), CGGTPGPQGIAGQRW (SEQ. ID 26), CGGMNYYSNS (SEQ. ID 27), CGGKKQRFRHRNRKG (SEQ. ID 28), CRGDGGGGGGGGGGGGGPHSRN (SEQ. ID 29), CPHSRNSGSGSGSGSGRGD (SEQ. ID 30), acetylated-GCYGRGDSPG (SEQ. ID 31), ((GPP)5GPC) (SEQ. ID 32), CRDGS (SEQ. ID 33), cyclic RGD{Fd}C (SEQ. ID 34), CGGRKRLQVQLSIRT (SEQ. ID 35), CIKVAV (SEQ. ID 36), CGGAASIKVAVSADR (SEQ. ID 37), CGGKRTGQYKL (SEQ. ID 38), CGGTYRSRKY (SEQ. ID 39), CGGYGGGP(GPP)5GFOGERPP(GPP)4GPC (SEQ. ID 40), CGGKRTGQYKLGSKTGPGQK (SEQ. ID 41), QAKHKQRKRLKSSC (SEQ. ID 42), SPKHHSQRARKKKNKNC (SEQ. ID 43), CGGXBBXBX, wherein B=basic residue and X=hydropathic residue (SEQ. ID 44), and CGGXBBBXXBX, wherein B=basic residue and X=hydropathic residue (SEQ. ID 45), or a combination thereof.

37. The method of claim 33, wherein R^{b} is a nucleic acid, polysaccharide, protein, lipid, tracer compound, aptamer, steroid, signaling molecule, or combinations thereof.

38. The method of claim 33, wherein X² is in each case independently selected from null, C(═O), CH₂CH₂, CH₂CH₂NH, or CH₂CH₂NHC(═O); and wherein Z² is null, C(═O), CH₂CH₂, CH₂CH₂NH, or CH₂CH₂NHC(═O).

39. The method of claim 33, wherein NB-core has the formula: 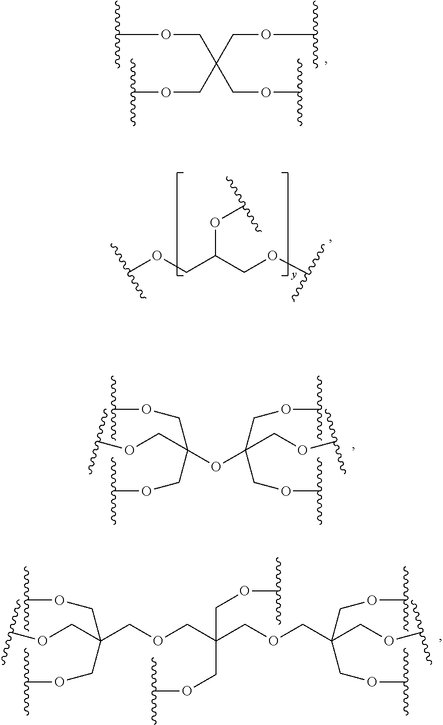
wherein y is 1-6.

40. The method of claim 33, wherein X¹ is (CH₂CH₂O)ₓ, wherein x is in each case independently selected from 1-500; and Z¹ is in each case independently selected from null or (CH₂CH₂O)_{z}, wherein z is in each case independently selected from 1-500,

41. The method of claim 33, wherein X² is in each case C(═O,) and Z² is null or CH₂CH₂.

42. The method of claim 33, wherein TH-core has the formula: 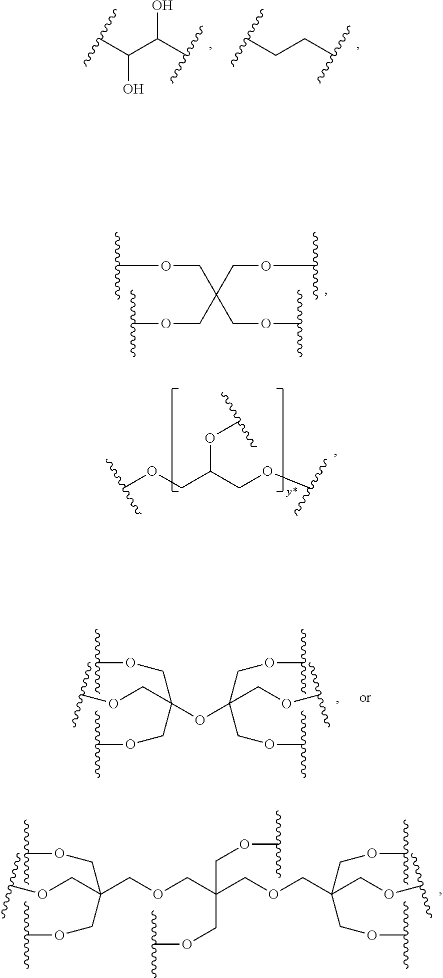
wherein y* is 1-6.

43. The method of claim 33, wherein the multi-armed norbornenyl compound is present in the composition in an amount from 1-25 wt. %.

44. The method of claim 33, wherein the composition comprises the thiolated adhesion peptide and multi-arm thiol compound in a molar ratio from 1:1 to 1:20.

45. The method of claim 33, wherein the crosslinked network defines a region within a microfluidic device, wherein the region comprises at least one reservoir in fluid communication with a channel.

46. The method of claim 33, wherein selectively irradiating the composition comprises providing a mask between the aqueous composition and a light source, the mask comprising a transparent portion and an opaque portion, the opaque portion defining at least one channel.

47. A microfluidic device comprising a top substrate layer, a bottom substrate layer, and a microfluidic layer disposed between the top and bottom substrate layers, wherein the microfluidic layer comprises a crosslinked hydrogel composition prepared according to claim 33.

48. The microfluidic device according to claim 47, wherein the microfluidic layer comprises a bottom surface contacting the bottom substrate layer and a top surface contacting the top substrate layer device, and at least one channel extending from the bottom surface to the top surface.

49. The microfluidic device according to claim 48, comprising a plurality of channels, wherein each channel is in fluid communication with at least two ports.

50. The microfluidic device according to claim 49, wherein the at least two ports comprises at least one inlet port, and at least one outlet port, wherein each channel is in fluid communication with the separate inlet port, and each channel is in fluid communication with the same outlet port; or each channel is in fluid communication the same inlet port and the same outlet port.

51. The microfluidic device according to claim 49, wherein each channel has a width that does not differ by more than 10% over the length of said channel.

52. A method of making a cellular structure, comprising depositing a cell, cell precursor, or combination thereof into a channel of the microfluidic device according to claim 47, and perfusing the channel with a nutrient.


SEQUENCE LISTING (45 sequences)

SEQ ID NO: 1 - protein, 7 aa, synthetic construct
  REGION 1..7: note = Description of sequence: component of photoactive peptide
GRGDSPC

SEQ ID NO: 2 - protein, 5 aa, synthetic construct
  REGION 1..5: note = Description of sequence: component of photoactive peptide
CRGDS

SEQ ID NO: 3 - protein, 6 aa, synthetic construct
  REGION 1..6: note = Description of sequence: component of photoactive peptide
CRGDSP

SEQ ID NO: 4 - protein, 6 aa, synthetic construct
  REGION 1..6: note = Description of sequence: component of photoactive peptide
CPHSRN

SEQ ID NO: 5 - protein, 10 aa, synthetic construct
  REGION 1..10: note = Description of sequence: component of photoactive peptide
CGWGGRGDSP

SEQ ID NO: 6 - protein, 16 aa, synthetic construct
  REGION 1..16: note = Description of sequence: component of photoactive peptide
CGGSIDQVEPYSSTAQ

SEQ ID NO: 7 - protein, 13 aa, synthetic construct
  REGION 1..13: note = Description of sequence: component of photoactive peptide
CGGRNIAEIIKDI

SEQ ID NO: 8 - protein, 12 aa, synthetic construct
  REGION 1..12: note = Description of sequence: component of photoactive peptide
CGGDITYVRLKF

SEQ ID NO: 9 - protein, 12 aa, synthetic construct
  REGION 1..12: note = Description of sequence: component of photoactive peptide
CGGDITVTLNRL

SEQ ID NO: 10 - protein, 10 aa, synthetic construct
  REGION 1..10: note = Description of sequence: component of photoactive peptide
CGGRYVVLPR

SEQ ID NO: 11 - protein, 15 aa, synthetic construct
  REGION 1..15: note = Description of sequence: component of photoactive peptide
CGGKAFDITYVRLKF

SEQ ID NO: 12 - protein, 14 aa, synthetic construct
  REGION 1..14: note = Description of sequence: component of photoactive peptide
CGGEGYGEGYIGSR

SEQ ID NO: 13 - protein, 23 aa, synthetic construct
  REGION 1..23: note = Description of sequence: component of photoactive peptide
  SITE 16: note = X= Nle
CGGATLQLQEGRLHFXFDLGKGR

SEQ ID NO: 14 - protein, 15 aa, synthetic construct
  REGION 1..15: note = Description of sequence: component of photoactive peptide
CGGSYWYRIEASRTG

SEQ ID NO: 15 - protein, 17 aa, synthetic construct
  REGION 1..17: note = Description of sequence: component of photoactive peptide
CGGGEFYFDLRLKGDKY

SEQ ID NO: 16 - protein, 16 aa, synthetic construct
  REGION 1..16: note = Description of sequence: component of photoactive peptide
CKGGNGEPRGDTYRAY

SEQ ID NO: 17 - protein, 16 aa, synthetic construct
  REGION 1..16: note = Description of sequence: component of photoactive peptide
CKGGPQVTRGDVFTMP

SEQ ID NO: 18 - protein, 15 aa, synthetic construct
  REGION 1..15: note = Description of sequence: component of photoactive peptide
CGGNRWHSIYITRFG

SEQ ID NO: 19 - protein, 14 aa, synthetic construct
  REGION 1..14: note = Description of sequence: component of photoactive peptide
CGGASIKVAVSADR

SEQ ID NO: 20 - protein, 11 aa, synthetic construct
  REGION 1..11: note = Description of sequence: component of photoactive peptide
CGGTTVKYIFR

SEQ ID NO: 21 - protein, 12 aa, synthetic construct
  REGION 1..12: note = Description of sequence: component of photoactive peptide
CGGSIKIRGTYS

SEQ ID NO: 22 - protein, 9 aa, synthetic construct
  REGION 1..9: note = Description of sequence: component of photoactive peptide
CGGSINNNR

SEQ ID NO: 23 - protein, 12 aa, synthetic construct
  REGION 1..12: note = Description of sequence: component of photoactive peptide
CGGSDPGYIGSR

SEQ ID NO: 24 - protein, 6 aa, synthetic construct
  REGION 1..6: note = Description of sequence: component of photoactive peptide
CYIGSR

SEQ ID NO: 25 - protein, 16 aa, synthetic construct
  REGION 1..16: note = Description of sequence: component of photoactive peptide
CGGTPGPQGIAGQGVV

SEQ ID NO: 26 - protein, 16 aa, synthetic construct
  REGION 1..16: note = Description of sequence: component of photoactive peptide
CGGTPGPQGIAGQRVV

SEQ ID NO: 27 - protein, 10 aa, synthetic construct
  REGION 1..10: note = Description of sequence: component of photoactive peptide
CGGMNYYSNS

SEQ ID NO: 28 - protein, 15 aa, synthetic construct
  REGION 1..15: note = Description of sequence: component of photoactive peptide
CGGKKQRFRHRNRKG

SEQ ID NO: 29 - protein, 22 aa, synthetic construct
  REGION 1..22: note = Description of sequence: component of photoactive peptide
CRGDGGGGGGGGGGGGGPHSRN

SEQ ID NO: 30 - protein, 19 aa, synthetic construct
  REGION 1..19: note = Description of sequence: component of photoactive peptide
CPHSRNSGSGSGSGSGRGD

SEQ ID NO: 31 - protein, 10 aa, synthetic construct
  REGION 1..10: note = Description of sequence: component of photoactive peptide
  MOD_RES 1: note = Amino acid is acetylated
GCYGRGDSPG

SEQ ID NO: 32 - protein, 18 aa, synthetic construct
  REGION 1..18: note = Description of sequence: component of photoactive peptide
GPPGPPGPPGPPGPPGPC

SEQ ID NO: 33 - protein, 5 aa, synthetic construct
  REGION 1..5: note = Description of sequence: component of photoactive peptide
CRDGS

SEQ ID NO: 34 - protein, 5 aa, synthetic construct
  REGION 1..5: note = Description of sequence: component of photoactive peptide
  CROSSLNK 1..5: note = cyclic bond
  SITE 5: note = d-phenylalanine
  REGION 1..5: note = peptide is circular
RGDFC

SEQ ID NO: 35 - protein, 15 aa, synthetic construct
  REGION 1..15: note = Description of sequence: component of photoactive peptide
CGGRKRLQVQLSIRT

SEQ ID NO: 36 - protein, 6 aa, synthetic construct
  REGION 1..6: note = Description of sequence: component of photoactive peptide
CIKVAV

SEQ ID NO: 37 - protein, 15 aa, synthetic construct
  REGION 1..15: note = Description of sequence: component of photoactive peptide
CGGAASIKVAVSADR

SEQ ID NO: 38 - protein, 11 aa, synthetic construct
  REGION 1..11: note = Description of sequence: component of photoactive peptide
CGGKRTGQYKL

SEQ ID NO: 39 - protein, 10 aa, synthetic construct
  REGION 1..10: note = Description of sequence: component of photoactive peptide
CGGTYRSRKY

SEQ ID NO: 40 - protein, 46 aa, synthetic construct
  REGION 1..46: note = Description of sequence: component of photoactive peptide
CGGYGGGPGPPGPPGPPGPPGPPGFOGERPPGPPGPPGPPGPPGPC

SEQ ID NO: 41 - protein, 20 aa, synthetic construct
  REGION 1..20: note = Description of sequence: component of photoactive peptide
CGGKRTGQYKLGSKTGPGQK

SEQ ID NO: 42 - protein, 14 aa, synthetic construct
  REGION 1..14: note = Description of sequence: component of photoactive peptide
QAKHKQRKRLKSSC

SEQ ID NO: 43 - protein, 17 aa, synthetic construct
  REGION 1..17: note = Description of sequence: component of photoactive peptide
SPKHHSQRARKKKNKNC

SEQ ID NO: 44-45 - skipped (2 entries)